(19) United States
(12) Patent Application Publication
(10) Pub. No.: US 2007/0185038 A1
(43) Pub. Date: Aug. 9, 2007

(54) Regulation of mammalian keratinous tissue using skin care actives

(75) Inventors: Donald Lynn Bissett, Hamilton, OH (US); Cheri Lynn Millikin, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/541,324

(22) Filed: Sep. 29, 2006

Related U.S. Application Data
(60) Provisional application No. 60/722,383, filed on Sep. 30, 2005.
(60) Provisional application No. 60/759,304, filed on Jan. 17, 2006.

(51) Int. Cl. A61K 31/075; A61K 31/13; A61K 31/155; A61K 31/16; A61K 31/34; A61K 31/355; A61K 31/44; A61K 31/7042

(52) U.S. Cl. 514/25; 514/355; 514/458; 514/474; 514/612; 514/628; 514/636; 514/709; 514/714

(57) ABSTRACT

Personal care compositions comprising skin and/or hair care actives. Such compositions are useful for regulating the condition of mammalian keratinous tissue needing such treatments, particularly skin lightening. In accordance with one embodiment, there is provided a personal care composition comprising a safe and effective amount of a first active selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, methyl sulfonyl methane, an avenathramide compound, and combinations thereof.

### CROSS-REFERENCE TO RELATED APPLICATIONS

This application claims the benefit of priority to U.S. Provisional Application Ser. No. 60/722,383, filed Sep. 30, 2005, and to U.S. Provisional Application Ser. No. 60/759,304, filed Jan. 17, 2006, both of which are herein incorporated by reference.

### TECHNICAL FIELD

The present invention relates to personal care compositions comprising skin and/or hair care actives. Such compositions are useful for regulating the condition of mammalian keratinous tissue needing such treatments, particularly skin lightening.

### BACKGROUND OF THE INVENTION

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling and histological changes typically associated with the aging of skin or environmental damage to human skin. However, there also exists a need for cosmetic agents to prevent, retard, and/or treat uneven skin tone by acting as a lightening or pigmentation reduction cosmetic agent.

Mammalian keratinous tissue, particularly human skin and hair, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage includes thinning of the skin, which occurs naturally as one ages. With such thinning, there is a reduction in the cells and blood vessels that supply the skin as well as a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990. Other damages or changes seen in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin (i.e., flaking, scaling, dryness, roughness). For hair, these extrinsic and intrinsic factors can contribute to, among other problems, hair bleaching, split ends, fragility, roughness, hair loss, reduction in hair growth rate, and the like. Therefore, there is a need for products and methods that seek to remedy these keratinous tissue conditions.

### SUMMARY OF THE INVENTION

The present invention relates to personal care compositions. Topical compositions that contain certain actives may be used to provide prophylactic as well as therapeutic treatments for keratinous tissue conditions. In accordance with one embodiment, there is provided a personal care composition comprising a safe and effective amount of a first active selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, methyl sulfonyl methane, an avenathramide compound, and combinations thereof. In a particular embodiment, the first active is selected from the group consisting of octadecenedioic acid, methyl sulfonyl methane, an avenathramide compound, and combinations thereof.

In accordance with another embodiment, the personal care composition also comprises a safe and effective amount of a second active, wherein said second active is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, and combinations thereof; and a safe and effective amount of a second active selected from the group consisting of hesperedin, mustard seed extract, glycyrrhizic acid, glycyrrhetinic acid, carnosine, Butylated Hydroxytoluene (BHT) and Butylated Hydroxyanisole (BHA), tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B₃ compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative (e.g., yeast cell extract), and combinations thereof.

In accordance with another embodiment, the personal care composition additionally comprises an additional component, preferably a safe and effective amount, wherein said additional component is selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof.

In still another embodiment, the personal care composition additionally comprises a dermatologically acceptable carrier.

The invention further relates to methods for regulating the condition of mammalian keratinous tissue, particularly for preventing, retarding, and/or treating uneven skin tone, wherein the methods each comprise the step of topically applying to the keratinous tissue of a mammal needing such treatment, a safe and effective amount of a personal care composition in accordance with the present invention.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential components as well as optional ingredients described herein.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application”, as used herein, means to apply or spread the compositions of the present invention onto the surface of the keratinous tissue.

The term “dermatologically acceptable,” as used herein, means that the compositions or components described are suitable for use in contact with human keratinous tissue without undue toxicity, incompatibility, instability, allergic response, and the like.

The term “safe and effective amount” as used herein means an amount of a compound or composition sufficient to induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “post-inflammatory hyperpigmentation” as used herein refers to the changes in melanin content as a response to an inflammatory event (e.g., acne, scratch, insect sting or bite, sunburn, etc), especially in dark skin subjects.

The term “hyperpigmentation” as used herein refers to an area of skin wherein the pigmentation is greater than that of an adjacent area of skin (e.g., a pigment spot, an age spot, and the like).

The terms “desquamation, exfoliation, and/or turnover” as used herein mean the removal of the upper layers of the stratum corneum (comprising the horny layers).

The terms “oily and/or shiny appearance” as used herein mean the glossy look mammalian skin tends to exhibit upon the excretion of oil, sebum, and/or sweat from the respective source gland.

The term “sagging” as used herein means the laxity, slackness, or the like condition of skin that occurs as a result of loss of, damage to, alterations to, and/or abnormalities in dermal elastin.

The term “smoothing” and “softening” as used herein means altering the surface of the keratinous tissue such that its tactile feel is improved.

The term “sallowness” as used herein means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

The compositions of the present invention are useful for topical application and for regulating keratinous tissue condition. Regulation of keratinous tissue condition, especially human skin condition, is often required due to conditions that may be induced or caused by factors internal and/or external to the body. For instance, “regulating skin condition” includes prophylactically regulating and/or therapeutically regulating skin condition, and may involve one or more of the following benefits: thickening (i.e., building the epidermis and/or dermis layers of the skin and/or the subcutaeous layers such as fat and muscle and where applicable the keratinous layers of the nail and hair shaft) to reduce atrophy (e.g., of the skin), increasing the convolution of the dermal-epidermal border, non-melanin skin discoloration such as under eye circles, blotching (e.g., uneven red coloration due to, e.g., rosacea) (hereinafter referred to as “red blotchiness”), sallowness (pale or yellow color), discoloration caused by telangiectasia or spider vessels, discolorations due to melanin (e.g., pigment spots, age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin. Regulating skin condition involves improving skin appearance and/or feel.

As used herein, “regulating skin condition” is intended to include regulation of such signs irrespective of the mechanism of origin.

### Components

First Actives

The present invention may include actives selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, methyl sulfonyl methane, an avenathramide compound, and combinations thereof. In a particular embodiment, the first active is selected from the group consisting of octadecenedioic acid, methyl sulfonyl methane, an avenathramide compound, and combinations thereof.

The actives of the present invention may be useful in skin lightening. Skin lightening may occur through multiple mechanisms including anti-oxidant mechanisms, trypsin inhibition, anti-inflammatory mechanisms, nitric oxide scavenging, tyrosinase inhibition, etc. Thus, compounds which have these mechanisms have the potential to lighten skin. Particular actives are discussed below in more detail.

### 1. Erythritol

The compositions of the present invention may include a safe and effective amount of erythritol (also known as “meso-erythritol”). When present, the composition contains erythritol in an amount from about 0.01% to about 10%, preferably from about 0.1% to about 5%, and more preferably from about 0.5% to about 3%, by weight of the total composition.

Erythritol is currently used as a sweetener, but has been discovered by Applicant to provide skin lightening potential. An erythritol useful herein can be described by the general structure shown below.

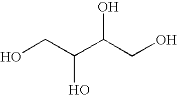

The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpigmented spots). Erythritol is believed to inhibit tyrosinase.

Erythtritol can be purchased from various suppliers, including Aldrich, Milwaukee, Wis., USA.

### 2. P-cymen-7-ol

The compositions of the present invention may include a safe and effective amount of p-cymen-7-ol (also known as “4-isopropyl benzyl alcohol”). When present, the composition contains p-cymen-7-ol in an amount from about 0.01% to about 10%, preferably from about 0.1% to about 5%, and more preferably from about 0.5% to about 3%, by weight of the total composition.

A p-cymen-7-ol useful herein can be described by the general structure shown below.

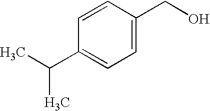

P-cymen-7-ol is believed to inhibit tyrosinase. P-cymen-7-ol can be purchased from various suppliers, including Aldrich, Milwaukee, Wis., USA.

### 3. Benzyl Phenylacetate

The compositions of the present invention may include a safe and effective amount of benzyl phenylacetate. When present, the composition contains benzyl phenylacetate in an amount from about 0.01% to about 10%, preferably from about 0.1% to about 5%, and more preferably from about 0.5% to about 3%, by weight of the total composition.

A benzyl phenylacetate useful herein can be described by the general structure shown below.

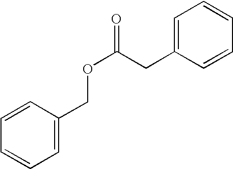

Benzyl phenylacetate is believed to inhibit tyrosinase. Benzyl phenylacetate can be purchased from various suppliers, including Aldrich, Milwaukee, Wis., USA.

### 4. 4-(4-methoxyphenyl)butan-2-one

The compositions of the present invention may include a safe and effective amount of 4-(4-methoxyphenyl)butan-2-one. When present, the composition contains 4-(4-methoxyphenyl)butan-2-one in an amount from about 0.01% to about 10%, preferably from about 0.1% to about 5%, and more preferably from about 0.5% to about 3%, by weight of the total composition.

The 4-(4-methoxyphenyl)butan-2-one useful herein can be described by the general structure shown below.

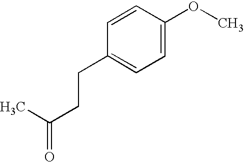

4-(4-methoxyphenyl)butan-2-one is believed to inhibit tyrosinase. 4-(4-methoxyphenyl)butan-2-one can be purchased from various suppliers, including Aldrich, Milwaukee, Wis., USA.

### 5. Ethoxyquin

The compositions of the present invention may include a safe and effective amount of ethoxyquin. When present, the composition contains ethoxyquin in an amount from about 0.01% to about 10%, preferably from about 0.1% to about 5%, and more preferably from about 0.5% to about 3%, by weight of the total composition.

An ethoxyquin useful herein can be described by the general structure shown below.

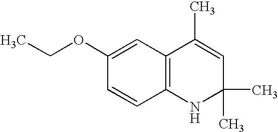

Variations of ethoxyquin that are equally useful herein include: 1,2-dihydro-6-ethoxy-2,2,4-trimethylquinoline and 6-ethoxy-1,2-dihydro-2,2,4-trimethylquinoline. Ethoxyquin is known as an anti-oxidant. Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. Since ethoxyquin has anti-oxidant properties, it can scavenge oxygen radicals before they stimulate the melanocytes. Ethoxyquin thus has skin lightening potential. Ethoxyquin can be purchased from various suppliers, including Sigma Chemical Company, St. Louis, Mo., USA.

### 6. Gallic Acid

The compositions of the present invention may include a safe and effective amount of gallic acid. When present, the composition contains gallic acid in an amount from about 0.01% to about 10%, preferably from about 0.1% to about 5%, and more preferably from about 0.5% to about 3%, by weight of the total composition. Gallic acid is also known as 3,4,5-trihydoxybenzoic acid.

Gallic acid can be described by the general structure shown below. Alternate forms of gallic acid include, for example, methyl gallate and trimethyl gallic acid, which are also shown below.

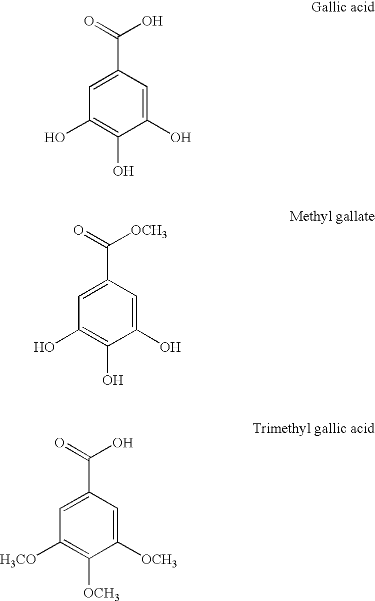

Gallic acid Methyl gallate Trimethyl gallic acid Gallic acid has been identified as an anti-oxidant with anti-inflammatory properties. Inflammatory mediators or cytokines can stimulate pigment cells (melanocytes) to produce melanin. Thus inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will in particular contribute to skin pigmentation in darker skin individuals (e.g., Hispanic, Asian). Inhibiting inflammation with anti-inflammatory agents will reduce pigmentation.

Gallic acid can be purchased from various suppliers, including Aldrich, Milwaukee, Wis., USA.

### 7. Tannic Acid

The compositions of the present invention may include a safe and effective amount of tannic acid. When present, the composition contains tannic acid in an amount from about 0.01% to about 10%, preferably from about 0.1% to about 5%, and more preferably from about 0.5% to about 3%, by weight of the total composition. Tannic acid is also known as gallotanic acid, digallic acid, allotannin, and tannimum.

A tannic acid useful herein can be described by the general structure shown below. As shown, there are four gallic acid molecules attached to a molecule of glucose, but tannic acids can contain up to eight gallic acid molecules.

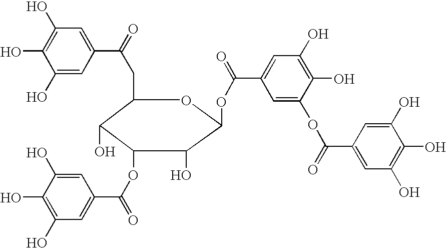

Tannic acid has been identified as an anti-oxidant with anti-inflammatory properties. Tannic acid can be purchased from various suppliers, including Aldrich, Milwaukee, Wis., USA.

### 8. Octadecenedioic acid

The compositions of the present invention may include a safe and effective amount of octadecenedioic acid. When present, the composition contains octadecenedioic acid in an amount from about 0.01% to about 10%, preferably from about 0.1% to about 5%, and more preferably from about 0.5% to about 3%, by weight of the total composition. Octadecenedioic acid is also known as C18:1 dicarboxylic acid and hexadec-8-ene-1,16-dioic acid.

An octadecenedioic acid useful herein can be described by the general structure shown below. 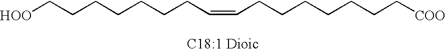
Octadecenedioic acid is PPAR-gamma agonist. PPARs (peroxisome proliferator-activated receptors) are a family of nuclear receptors (PPAR-alpha, -beta, -gamma) that regulate a number of cell functions. One such function, mediated by PPAR-gamma, is control of cell proliferation. Thus a material that binds to PPAR-gamma and acts as an agonist of that receptor will reduce proliferation which can include down-regulation of genes such as the gene for tyrosinase, leading to less production of the tyrosinase enzyme in the melanocyte. This would result in less production of melanin and thus lightening of skin color. Octodecenedioic acid can be purchased from the supplier Uniqema, New Castle, Del., USA. Arlatone Dioic DCA™ is a preferred octodecenedioic acid available from Uniqema. In this form of octodecenedioic acid, the double bond is between carbons 8 and 9.

### 9. P-cymen-5-ol

The compositions of the present invention may include a safe and effective amount of p-cymen-5-ol. When present, the composition contains p-cymen-5-ol in an amount from about 0.01% to about 10%, preferably from about 0.1% to about 5%, and more preferably from about 0.5% to about 3%, by weight of the total composition. This active is also known as para-thymol and as 3-methyl-4-(1-methylethyl)phenol.

The p-cymen-5-ol useful herein can be described by the general structure shown below.

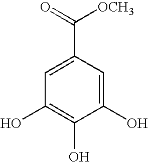

P-cymen-5-ol is believed to inhibit tyrosinase. It can be purchased from various suppliers, including Sigma Chemical Company, St. Louis, Mo., USA.

### 10. Methyl Sulfonyl Methane

The compositions of the present invention may include a safe and effective amount of methyl sulfonyl methane (also known as dimethyl sulfone). When present, the composition contains methyl sulfonyl methane in an amount from about 0.01% to about 10%, preferably from about 0.1% to about 5%, and more preferably from about 0.5% to about 3%, by weight of the total composition.

Methyl sulfonyl methane is a sulfur source for endogenous biosynthesis of the sulfur-containing intracellular anti-oxidant glutathione (gamma-glutamyl-cysteinyl-glycine). Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. Since glutathione has anti-oxidant properties, it can scavenge oxygen radicals before they stimulate the melanocytes. Methyl sulfonyl methane, as a precursor to the sulfur in the cysteine residue of glutathione, thus has skin lightening potential.

### 11. Avenanthramides

The compositions of the present invention may include a safe and effective amount of an avenathramide compound. When present, the composition contains the avenanthramide compound in an amount from about 0.01% to about 10%, and preferably in an amount from about 0. 1% to about 2%, by weight of the total composition.

Avenanthramide compounds have been identified as anti-histamines, anti-inflammatories, and anti-itch compounds. Avenanthramide compounds are commercially available from Symrise (Holzminden, Germany), sold under the tradename SymCalmin. Exemplary avenanthramide compounds are illustrated below. Preferred compounds include the dihydroavenanthramide compounds. 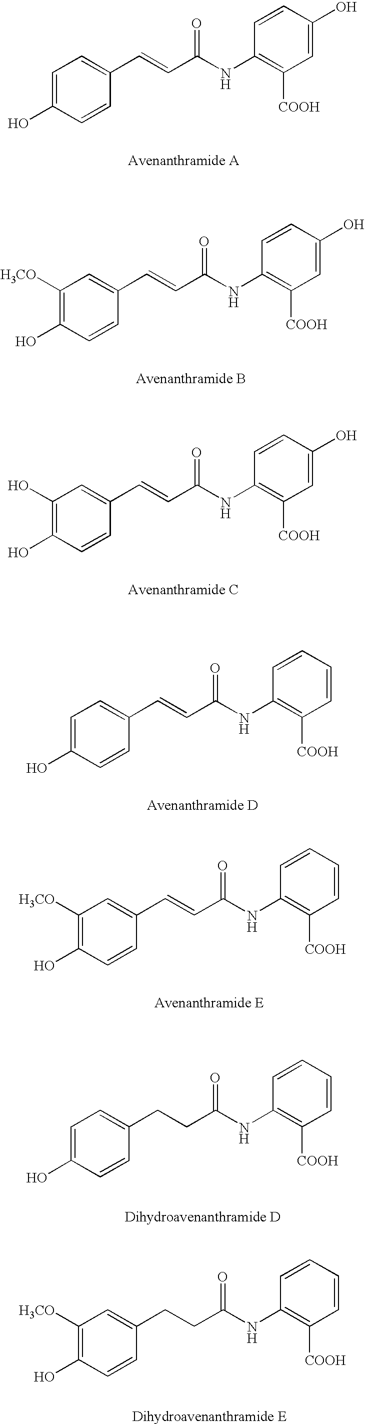 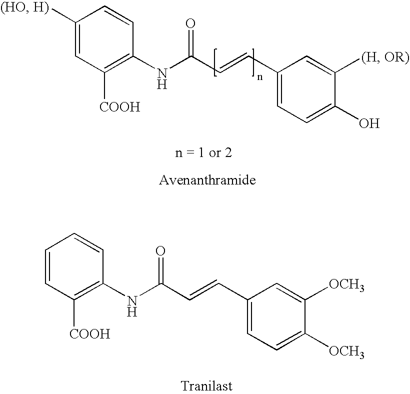
Additional Actives

The present invention may include additional actives selected from the group consisting of second active, wherein said second active is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, and combinations thereof; and a safe and effective amount of a second active selected from the group consisting of hesperedin, mustard seed extract, glycyrrhizic acid, glycyrrhetinic acid, carnosine, Butylated Hydroxytoluene (BHT) and Butylated Hydroxyanisole (BHA), tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B₃ compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative (e.g., yeast cell extract), and combinations thereof.

Particular additional actives are discussed in more detail below.

1. Sugar Amines (Amino Sugars)

The compositions of the present invention optionally include a safe and effective amount of a sugar amine, which are also known as amino sugars. The sugar amine compounds useful in the present invention are described in PCT Publication WO 02/076423 and U.S. Pat. No. 6,159,485.

Preferably, and when present, the composition contains from about 0.01% to about 15%, more preferably from about 0.1% to about 10%, and even more preferably from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

2. Vitamin B₃

The compositions of the present invention may include a safe and effective amount of a vitamin B₃ compound. Vitamin B₃ compounds are particularly useful for regulating skin condition as described in U.S. Pat. No. 5,939,082. Preferably, and when present, the composition contains from about 0.01% to about 50%, more preferably from about 0.1% to about 20%, even more preferably from about 0.5% to about 10%, and still more preferably from about 1% to about 7%, even more preferably from about 2% to about 5%, by weight of the composition, of the vitamin B₃ compound.

As used herein, “vitamin B₃ compound” means a compound having the formula: 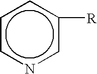
wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate, myristyl nicotinate).

Examples of suitable vitamin B₃ compounds are well known in the art and are commercially available from a number of sources (e.g., the Sigma Chemical Company, ICN Biomedicals, Inc., and Aldrich Chemical Company). A preferred vitamin B₃ compound useful in the present invention is niacinamide.

3. Retinoid

The compositions of this invention may contain a safe and effective amount of a retinoid, such that the resultant composition is safe and effective for regulating keratinous tissue condition, preferably for regulating visible and/or tactile discontinuities in skin, more preferably for regulating signs of skin aging. When present, the compositions preferably contain from about 0.001% to about 10%, more preferably from about 0.005% to about 2%, even more preferably from about 0.01% to about 1%, still more preferably from about 0.01% to about 0.5%, by weight of the composition, of the retinoid. The optimum concentration used in a composition will depend on the specific retinoid selected since their potency does vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. Preferred retinoids are retinol, retinyl palmitate, retinyl acetate, retinyl propionate, retinal and combinations thereof. More preferred is retinyl propionate, used even more preferably from about 0.1% to about 0.3%.

4. Peptide

The compositions of the present invention may contain a safe and effective amount of a peptide, including but not limited to, di-, tri-, tetra-, penta-, and hexa-peptides and derivatives thereof. When present, the compositions contain preferably from about 1×10⁻⁶% to about 20%, more preferably from about 1×10⁻⁶% to about 10%, even more preferably from about 1×10⁻⁵% to about 5%, by weight of the composition.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as lamin). A preferred dipeptide derivative is palmitoyl-lys-thr. A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmitoyl-lys-thr-thr-lys-ser and is commercially available from Sederma.

5. Phytosterol

The topical compositions of the present invention may comprise a safe and effective amount of one or more phytosterols selected from the group consisting of β-sitosterol, campesterol, brassicasterol, Δ5-avennasterol, lupenol, x-spinasterol, stigmasterol, their derivatives, analogs, and combinations thereof. More preferably, the phytosterol is selected from the group consisting of β-sitosterol, campesterol, brassicasterol, stigmasterol, their derivatives, and combinations thereof. More preferably, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

When present in the compositions, the phytosterol preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2% by weight of the composition. 6. Hexamidine Compounds

The topical compositions of the present invention optionally include a safe and effective amount of one or more of hexamidine compounds, which can include, but are not limited to, hexamidine and its salts and its derivatives. As used herein, hexamidine derivatives include any isomers and tautomers of hexamidine compounds including but not limited to organic acids and mineral acids, for example sulfonic acid, carboxylic acid etc. Preferably, the hexamidine compounds include hexamidine diisethionate, commercially available as Eleastabg HP100 from Laboratoires Serobiologiques.

The hexamidine compounds useful in the present invention correspond to those of the following chemical structure: 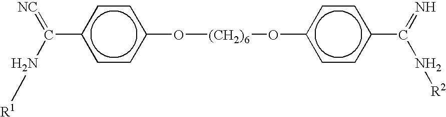
wherein R¹ and R² comprise organic acids (e.g., sulfonic acids, etc.).

When present in the composition, the hexamidine preferably comprises from about 0.0001 to about 25%, more preferably from about 0.001 to about 10%, more preferably from about 0.01 to about 5%, and even more preferably from about 0.02 to about 2.5% by weight of the composition. 7. Dialkanoyl Hydroxyproline Compounds

The topical compositions of the present invention may comprise a safe and effective amount of one or more dialkanoyl hydroxyproline compounds and their salts and derivatives. When present in the composition, the dialkanoyl hydroxyproline compounds preferably comprise from about 0.01 to 10%, more preferably from about 0.1-5%, even more preferably from about 0.1 to 2% by weight of the composition

The dialkanoyl hydroxyproline compounds of the present invention correspond to those of the following chemical structure: 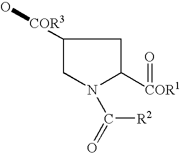
wherein R¹ comprises H, X, C₁-C₂₀ straight or branched alkyl,

X comprises metals (Na, K, Li, Mg, Ca) or amines (DEA, TEA);

R² comprises C₁-C₂₀ straight or branched alkyl;

R³ comprises C₁-C₂₀ straight or branched alkyl.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably the dipalmitoyl hydroxyproline is the triethanolamine salt of dipalmitoyl hydroxyproline.

8. Salicylic Acid Compound

The topical compositions of the present invention may comprise a safe and effective amount of a salicylic acid compound, its esters, its salts, or combinations thereof. When present in the compositions, the salicylic acid compound preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2%, by weight of the composition, of salicylic acid.

9. N-acyl Amino Acid Compound

The topical compositions of the present invention may comprise a safe and effective amount of one or more N-acyl amino acid compounds. The amino acid can be one of any of the amino acids known in the art. The N-acyl amino acid compounds of the present invention correspond to the formula: 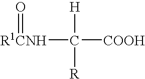
wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W.H. Freeman and Company. R¹ can be C₁ to C₃₀, saturated or unsaturated, straight or branched, substituted or unsubstituted alkyls; substituted or unsubstituted aromatic groups; or mixtures thereof.

Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula: 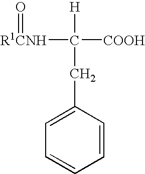
wherein R¹ can be C₁ to C₃₀, saturated or unsaturated, straight or branched, substituted or unsubstituted alkyls; substituted or unsubstituted aromatic groups; or mixtures thereof.

N-acyl Tyrosine corresponds to the following formula: 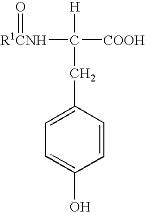
wherein R¹ can be C₁ to C₃₀, saturated or unsaturated, straight or branched, substituted or unsubstituted alkyls; substituted or unsubstituted aromatic groups; or mixtures thereof.

Particularly useful as a topical skin tone evening (lightening or pigmentation reduction) cosmetic agent is N-undecylenoyl-L-phenylalanine. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

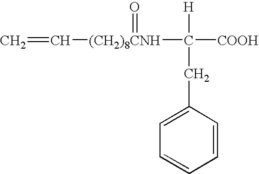

As used herein, N-undecylenoyl-L-phenylalanine is commercially available under the tradename Sepiwhite® from SEPPIC.

When present in the compositions, the N-acyl amino acid preferably comprises from about 0.0001-25%, more preferably from about 0.001-10%, more preferably from about 0.01-5%, and even more preferably from about 0.02-2.5% by weight of the composition.

10. Sunscreen Actives

The compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Particularly suitable sunscreen agents are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Preferred organic sunscreen actives useful in the compositions of the present invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

The sunscreen active preferably comprises from about 1% to about 20%, more preferably from about 2% to about 10%, by weight of the composition when present. Exact amounts will vary depending upon the sunscreen chosen and the desired Sun Protection Factor (SPF).

11. Water-Soluble Vitamins

The compositions of the present invention may contain a safe and effective amount of one or more water-soluble vitamins. Examples of water-soluble vitamins include, but are not limited to, water-soluble versions of vitamin B (such as vitamin B5 and vitamin B6), vitamin B derivatives, vitamin C compounds (including compounds such as ascorbyl glucoside, and including vitamin C derivatives such as magnesium ascorbyl phosphate, sodium ascorbyl phosphate, and ascorbyl palmitate), vitamin K, vitamin K derivatives, pro-vitamins thereof, such as panthenol and mixtures thereof. When vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the vitamin compound.

12. Oil-Soluble Vitamins

The compositions of the present invention may contain a safe and effective amount of one or more oil-soluble vitamins. Examples of oil-soluble vitamins include, but are not limited to, oil-soluble versions of vitamin D, vitamin D derivatives, vitamin E (such as vitamin E acetate), vitamin E derivatives, pro-vitamins thereof, and mixtures thereof. When oil-soluble vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the oil-soluble vitamin compound.

13. Hesperedin

The compositions of the present invention may include a safe and effective amount of hesperedin. Preferably, the composition contains from about 0.01% to about 10%, more preferably from about 0.1% to about 5%, even more preferably from about 0.5% to about 3%, by weight of the composition, of the hesperedin compound when present.

Hesperedin is a flavonoid. Oxygen radicals are produced in the skin in response to many stimuli, such as exposure to UV and irritatants. Such radicals are also produced as by-products of normal cell or tissue metabolism. Oxygen radicals can stimulate pigment cells (melanocytes) to increase production of melanin. Hesperidin has anti-oxidant properties and thus can scavenge oxygen radicals before they stimulate the melanocytes. Hesperidin also inhibits tyrosinase.

14. Mustard Seed Extract

The compositions of the present invention may include a safe and effective amount of mustard seed extract. When present, the composition preferably contains from about 0. 1% to about 20%, more preferably from about 0.5% to about 10%, even more preferably from about 1% to about 5%, by weight of the composition, of the mustard seed extract compound. A preferred mustard seed extract is Sinablancag. Sinablanca®V is hydrolyzed Brassica Alba seed extract and is believed to inhibit tyrosinase

15. Glycyrrhizic acid

The compositions of the present invention may include a safe and effective amount of glycyrrhizic acid. When present, the composition preferably contains from about 0.01% to about 10%, more preferably from about 0.05% to about 5%, even more preferably from about 0.1% to about 3%, by weight of the composition, of the glycyrrhizic acid compound. Glycyrrhizic acid is a component of licorice extract.

Glycyrrhizic acid is an anti-inflammatory agent. Inflammatory mediators or cytokines can stimulate pigment cells (melanocytes) to produce melanin. Thus inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will in particular contribute to skin pigmentation in darker skin individuals (e.g., Hispanic, Asian). Inhibiting inflammation with anti-inflammatory agents will reduce pigmentation.

Glycyrrhizic acid is also believed to be a scavenger of nitric oxide. Nitric oxide (NO) is a stimulator of pigmentation. Use of nitric oxide scavengers (materials that react with nitric oxide to prevent it from stimulating pigment cells) will reduce pigmentation.

Glycyrrhizic acid is also known as glycyrrhizin, glycyrrhizinic acid, or glycyrrhetinic acid glycoside.

16. Glycyrrhetinic acid

The compositions of the present invention may include a safe and effective amount of glycyrrhetinic acid. When present, the composition preferably contains from about 0.01% to about 10%, more preferably from about 0.05% to about 5%, even more preferably from about 0.1% to about 3%, by weight of the composition, of the glycyrrhetinic acid compound. Glycyrrhetinic acid is a component of licorice extract.

Glycyrrhetinic acid is also an anti-inflammatory agent, discussed above in the glycyrrhizic acid section. Structurally, glycyrrhetinic acid is different from glycyrrhizic acid in that glycyrrhetinic acid does not have an attached sugar residue (glycoside). Glycyrrhetinic acid is also known as enoxolone, glycyrrhetic acid, or uralenic acid.

17. Carnosine

The compositions of the present invention may include a safe and effective amount of carnosine. When present, the composition preferably contains from about 0.01% to about 20%, more preferably from about 0.1% to about 15%, even more preferably from about 1% to about 10%, by weight of the composition, of the carnosine compound.

Carnosine is a dipeptide and acts as an anti-oxidant. The anti-oxidant mechanism is the same as that described above in hesperidin section. Carnosine is found naturally in the human body. It has been called the anti-aging peptide since it is present in high levels in longer-lived tissues and is present at low levels in tissues with issues (e.g., cataracts). Materials that are structurally and mechanistically similar to carnosine include carcinine, anserrine, homocarnosine and ophidine.

18. Butylated Hydroxytoluene (BHT) and Butylated Hydroxyanisole (BHA)

The compositions of the present invention may include a safe and effective amount of BHT or BHA. The BHT useful herein can be described by the general structure: 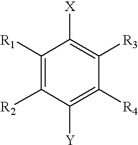
wherein X is selected from the group consisting of OH and SH; Y is selected from the group consisting of H, OH, OR₅, COOR₅, alkyl, cycloalkyl, heteroalkyl, heterocycloalkyl, aromatic, heteroaromatic, carboxamido, sulfonamido, carbamate, urea, and trialkylsilyl; R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen; R₅ is selected from the group consisting of alkyl, cycloalkyl, heteroalkyl, heterocycloalkyl, aromatic, heteroaromatic, trialkylsilyl, acyl, and hydrogen.

BHT is commonly used as a preservative of products because of its anti-oxidant properties, but higher doses may have skin lightening properties. BHA and BHT can be purchased from various suppliers, including Eastman Chemical (Kingsport, Tenn.), Alfa Chemical (Kings Point, N.Y.), and Shell Chemical Company (Houston, Tex.).

BHT or BHA may be present in an amount of from about 0.01% to about 10%, more preferably from about 0.05% to about 5%, more preferably from about 0.1% to about 3%, by weight of the composition.

19. Tetrahydrocurcumin

The compositions of the present invention may include a safe and effective amount of tetrahydrocurcumin, its esters (e.g., diacetate ester), or combinations of these. When present, the composition preferably contains from about 0.01% to about 10%, more preferably from about 0.1% to about 5%, even more preferably from about 0.25% to about 3%, by weight of the composition, of the tetrahydrocurcumin compound.

Tetrahydrocurcumin is known for its anti-oxidant and tyrosinase inhibition properties through the mechanisms discussed above.

20. Cetyl Pyridinium Chloride (CPC)

The compositions of the present invention may comprise a safe and effective amount of cetyl pyridinium chloride (CPC). Alternate forms of cetyl pyridinium chloride include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quatemized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215.

Cetyl pyridinium chloride may be present in an amount of from about 0.005% to about 10% by weight of the composition, more preferably from about 0.01% to about 5%, more preferably from about 0.05% to about 2%. Cetyl pyridinium chloride is an inhibitor of tyrosinase, a mechanism discussed above.

21. Ergothioneine

The compositions of the present invention may comprise a safe and effective amount of ergothioneine. Ergothioneine may be present in an amount of from about 0.01% to about 20% by weight of the composition, more preferably from about 0.1% to about 15% by weight of the composition, even more preferably from about 1% to about 10% by weight of the composition. A preferred ergothioneine is Thiotaine( which is a commercial solution of the chemical ergothioneine, commercially available from Barnet Products. Ergothioneine exhibits anti-oxidant properties, a mechanism described above.

22. Vanillin

The compositions of the present invention may comprise a safe and effective amount of vanillin or its derivatives. Vanillin may be present in an amount of from about 0.01% to about 20% by weight of the composition, more preferably from about 0.1% to about 15% by weight of the composition, even more preferably from about 0.5% to about 10% by weight of the composition.

A preferred vanillin derivative is vanillin acetate, which is believed to be a subtilisin-type protease inhibitor. The peptide hormone melanocyte stimulating hormone (MSH) induces pigment cells to make melanin. MSH is produced as a larger inactive precursor peptide (pro-hormone) which must be cleaved by a protease to the active MSH. That protease is a subtilisin-type protease.

Other related compounds useful in the present invention include vanillic acid, vanillin, o-vanillin, ethyl vanillin, isovanillin, vanillin methyl ether, vanillin ethyl ether, o-ethyl vanillin, vanillin oxime, vanillin benzyl ether, homovanillin, vanillin isobutyrate, divanillin, and isovanillin oxime.

23. Diethylhexyl Syrinylidene Malonate

The compositions of the present invention may comprise a safe and effective amount of diethylhexyl syrinylidene malonate. Diethylhexyl syrinylidene malonate may be present in an amount of from about 0.01% to about 20% by weight of the composition, more preferably from about 0.1% to about 15% by weight of the composition, even more preferably from about 0.5% to about 10% by weight of the composition.

A preferred diethylhexyl syrinylidene malonate is Oxynex® which exhibits anti-oxidant properties. It is available from Rona/Merck.

24. Melanostatine

The compositions of the present invention may comprise a safe and effective amount of melanostatine. Melanostatine may be present in an amount of from about 0.01% to about 20% by weight of the composition, more preferably from about 0.1% to about 15% by weight of the composition, even more preferably from about 0.5% to about 10% by weight of the composition. Since melanostatine is a commercial solution of peptide (approximately 50 ppm peptide in this commercial solution), the actual level of peptide in a product containing 5% melanostatine actually contains approximately 2.5 ppm peptide).

A preferred melanostatine is available from Vincience (France). Melanostatine is a hexapeptide, and it operates mechanistically by inhibiting binding of alpha-MSH (melanin stimulating hormone) to its cell receptor, thus inhibiting initiation of pigmentation.

25. Sterol Esters

The compositions of the present invention may comprise a safe and effective amount of sterol esters. The sterol esters may be present in an amount of from about 0.01% to about 20% by weight of the composition, more preferably from about 0.1% to about 15% by weight of the composition, even more preferably from about 0.5% to about 10% by weight of the composition.

When sterol esters are used in the present invention, formulation of the composition should be performed so that hydrolysis of the esters does not occur. Therefore, the ideal pH range of the composition comprising sterol esters is from about 3 to about 8, preferably from about 4 to about 7.

Sterol esters useful in the present invention may be comprised of sterols or mixtures of sterols (in particular sitosterol, campesterol, stigmasterol, brassicasterol, and additional sterols) which are esterified with a fatty acid or mixtures of fatty acids (which can be straight chain or branched chain, saturated or unsatured) with from 8 to 30 carbon atoms (preferably 16-22 carbon atoms). Sterol esters are available from P&G Chemicals.

26. Creatine and Creatinine

The compositions of the present invention may comprise a safe and effective amount of creatine, a creatine derivative, creatinine, or combinations thereof. Creatine, creatine derivatives, or creatinine may be present in an amount of from about 0.01% to about 20% by weight of the composition, preferably from about 0.1% to about 15% by weight of the composition, and more preferably from about 1% to about 10% by weight of the composition.

Creatine derivatives include, but are not limited to, creatine phosphate, creatine sulfate, creatine acetate, creatine ascorbate and derivatives esterified on the carboxyl group with mono- or polyfunctional alcohols. Creatine phosphate is one preferred creatine derivative, and has the structure shown below.

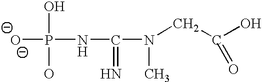

Creatinine is characterized by the structure shown below.

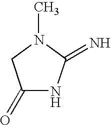

27. Feverfew Extract

The compositions of the present invention may comprise a safe and effective amount of a feverfew extract, in an amount of from about 0.01% to about 20% by weight of the composition, preferably from about 0.1% to about 15% by weight of the composition, and more preferably from about 1% to about 10% by weight of the composition.

28. Licochalcone A

The compositions of the present invention may comprise a safe and effective amount of an extract of radix glycyrrhizae inflatae containing licochalcone A, in an amount of from about 0.01% to about 20% by weight of the composition, preferably from about 0.1% to about 15% by weight of the composition, and more preferably from about 1% to about 10% by weight of the composition.

One constituent of the aqueous extract of radix glycyrrhizae inflatae is licochalcone A, which is characterized by the below structural formula.

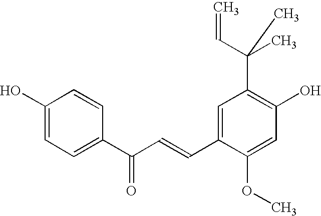

29. Yeast Cell Derivatives

Any suitable yeast cell derivative, such as yeast extract, can be used herein. For instance, yeast extract can be an extract of yeast cells and/or the culture fluid remaining after growth of yeast. The extract solvent can be any suitable solvent for solubilizing yeast cell or culture fluid components, such as water, alcohol, or glycol. In one embodiment, the solvent is water. An example of a suitable yeast extract is Saccharomycopsis Ferment Filtrate, available under the trade name Pitera™ from Kashiwayama.

Dermatologically Acceptable Carrier

The topical compositions of the present invention also comprise a dermatologically acceptable carrier for the active materials. The phrase “dermatologically acceptable carrier”, as used herein, means that the carrier is suitable for topical application to the keratinous tissue, has good aesthetic properties, is compatible with the actives of the present invention and any other components, and will not cause any safety or toxicity concerns.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, can be useful herein.

The compositions of the present invention can also comprise other dermatologically acceptable topical carrierss. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

Additional Components

The compositions of the present invention may contain any other suitable components that are desired. For instance, the compositions can include a variety of other ingredients that are conventionally used in given product types provided that they do not unacceptably alter the benefits of the invention. In one embodiment, an additional component, preferably a safe and effective amount, is selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof.

### Composition Forms

The topical compositions of the subject invention can include, but are not limited to, cleaners, lotions, milks, mousses, serums, sprays, aerosols, foams, sticks, pencils, gels, creams and ointments. The compositions can be, for example, formulated as toilet bars, liquids, shampoos, bath gels, hair conditioners, hair tonics, pastes, or mousses. The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which can be suitable for use herein are described, for example, in U.S. Pat. No. 6,060,547.

### Composition Preparation

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Methods for Regulating Keratinous Tissue Condition

The compositions of the present invention are useful for regulating a number of mammalian keratinous tissue conditions. Such regulation of keratinous tissue conditions includes prophylactic and therapeutic regulation. More specifically, such regulating methods are directed to, but are not limited to, thickening keratinous tissue (i.e., building the epidermis and/or dermis and/or subcutaneous layers of the skin and where applicable the keratinous layers of the nail and hair shaft), preventing, retarding, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of mammalian skin, desquamating, exfoliating, and/or increasing turnover in mammalian skin, reducing the size of pores in mammalian skin, regulating oily/shiny appearance of mammalian skin, preventing, retarding, and/or treating hyperpigmentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin. Applicants have surprisingly found that compositions of the present invention are useful for the above disclosed methods as well.

Regulating keratinous tissue condition involves topically applying to the keratinous tissue a safe and effective amount of a composition of the present invention. The amount of the composition that is applied, the frequency of application and the period of use will vary widely depending upon the level of skin care actives and/or other components of a given composition and the level of regulation desired.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application are, in mg composition/cm² skin, from about 0.1 mg/cm² to about 20 mg/cm². A particularly useful application amount is about 0.5 mg/cm² to about 10 mg/cm².

Treating keratinous tissue condition can be practiced, for example, by applying a composition in the form of a skin lotion, clear lotion, milky lotion, cream, gel, foam, ointment, paste, emulsion, spray, aerosol, conditioner, tonic, cosmetic, lipstick, foundation, nail polish, after-shave, roll-on or deodorant stick, powder, oil or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.) The composition can be dispensed from a bottle, jar, tube, sachet, pouch, container, tottle, vial, ampule, compact, etc. or can be integrally contained within a delivery form such as a wipe. The application of the present compositions may be done using the palms of the hands and/or fingers. The application may also be done with the aid of a device or implement such as a cotton ball, swab, pad, brush, eye dropper, puff, sponge, wand, wipe, foam, nonwoven substrate, mask, roll-on applicator, stick applicator, applicator pen, spray applicator, atomizer, razor, etc. The active may be contained in a rupturable pouch between two substrates.

In another embodiment, the application of the topical composition is subsequent to a skin treatment such as cleansing, exfoliation or tanning.

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, upper lip, and the like). The patch can be occlusive, semi-occlusive or non-occlusive, and can be adhesive or non-adhesive. The composition can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can also contain a source of electrical energy (e.g., a battery) to, for example, increase delivery of the composition and active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

Other devices can also be employed in conduction with use of the actives of the present invention. For example, ultrasound, lasers, heating devices, and the like can be employed to enhance the benefits for skin and hair.

Another approach to enhancing the benefits of the actives is use of a kit or regimen of 2 or 3 or 4 or more products and/or treatment procedures (e.g., exfoliation followed by topical treatment with one or more of the actives of the present invention, depilation of hair followed by topical treatment with one or more of the actives of the present invention, and the like). The various components of a regimen can be used in a short period of time (e.g., within an hour) or spread over a longer time frame within a day (e.g., morning and evening) or over even longer time periods (e.g., one step in the regimen done weekly or monthly and the other steps in the regimen done on a more regular basis, e.g., daily).

Combinations of an oral composition and a topical composition can be packaged together as a kit. In another embodiment, the oral composition and the topical composition are not packaged together as a kit, but potential users of the regimen are informed (e.g. through advertisements, product labeling) that the oral and the topical compositions may be used in conjunction with one another to regulate the condition of kerationous tissue.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples A, B, C and D

### Moisturizing Lotions/Creams

| Component | A | B | C | D |
|---|---|---|---|---|
| Disodium EDTA | 0.100 | 0.100 | 0.100 | 0.100 |
| Dihydrovenanthramide D | 2.000 | 0 | 0 | 0 |
| Dihydrovenanthramide E | 0 | 2.000 | 0 | 0 |
| Methyl sulfonyl methane | 0 | 0 | 2.000 | 0 |
| Niacinamide | 4.000 | 4.000 | 4.000 | 5.000 |
| Octadecenedioic acid | 0 | 0 | 0 | 2.000 |
| Isohexadecane | 3.000 | 3.000 | 3.000 | 3.000 |
| Isopropyl isostearate | 1.330 | 1.330 | 1.330 | 1.330 |
| Sucrose polycottonseedate | 0.670 | 0.670 | 0.670 | 0.670 |
| Polymethylsilsesquioxane | 0.250 | 0.250 | 0.250 | 0.250 |
| Cetearyl glucoside + cetearyl | 0.200 | 0.200 | 0.200 | 0.200 |
| alcohol | | | | |
| Behenyl alcohol | 0.400 | 0.400 | 0.400 | 0.400 |
| Ethylparaben | 0.200 | 0.200 | 0.200 | 0.200 |
| Propylparaben | 0.100 | 0.100 | 0.100 | 0.100 |
| Cetyl alcohol | 0.320 | 0.320 | 0.320 | 0.320 |
| Stearyl alcohol | 0.480 | 0.480 | 0.480 | 0.480 |
| Tocopheryl acetate | 0.500 | 0.500 | 0.500 | 0.500 |
| PEG-100 stearate | 0.100 | 0.100 | 0.100 | 0.100 |
| Glycerin | 7.000 | 7.000 | 7.000 | 10.000 |
| Titanium dioxide | 0.604 | 0.604 | 0.604 | 0.604 |
| Polyacrylamide + C13-14 | 2.000 | 2.000 | 2.000 | 2.000 |
| isoparaffin + laureth-7 | | | | |
| Panthenol | 1.000 | 1.000 | 1.000 | 1.000 |
| Benzyl alcohol | 0.400 | 0.400 | 0.400 | 0.400 |
| Dimethicone + dimethiconol | 2.000 | 2.000 | 2.000 | 2.000 |
| Water (to 100 g) | to | to | to | To |
| | 100 | 100 | 100 | 100 |
| TOTAL | 100 | 100 | 100 | 100 |

In a suitable vessel, combine the water phase ingredients and heat to 75° C. In a separate suitable vessel, combine the oil phase ingredients and heat to 75° C. Next, add the oil phase to the water phase and mill the resulting emulsion (e.g., with a Tekmar™ T-25 mill). Then, add the thickener to the emulsion and cool the emulsion to 45° C. while stirring. At 45° C., add the remaining ingredients. Cool the product and stir to 30° C. and pour into suitable containers.

The compositions are chronically applied topically to areas of hyperpigmented skin. Versus a control composition, the compositions of the examples show a statistically significant skin-lightening benefit.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition, comprising:
a. a safe and effective amount of a first active selected from the group consisting of octadecenedioic acid, methyl sulfonyl methane, an avenathramide compound, and combinations thereof;
b. optionally, a safe and effective amount of a second active, wherein said second active is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, and combinations thereof; and a safe and effective amount of a second active selected from the group consisting of hesperedin, mustard seed extract, glycyrrhizic acid, glycyrrhetinic acid, camosine, Butylated Hydroxytoluene (BHT) and Butylated Hydroxyanisole (BHA), tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B₃ compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative, and combinations thereof;
c. optionally, an additional component, wherein said additional component is selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof; and
d. optionally, a dermatologically acceptable carrier.

2. The personal care composition of claim 1, wherein said composition comprises said second active.

3. The personal care composition of claim 2, wherein said second active is selected from the group consisting of vitamin B₃ compounds, water soluble vitamins, oil-soluble vitamins, hexamidine compounds, and combinations thereof.

4. The personal care composition of claim 3, wherein said vitamin B₃ compound comprises niacinamide; said water soluble vitamin is selected from the group consisting of a vitamin C compound and panthenol; and said oil-soluble vitamin comprises vitamin E acetate.

5. The personal care composition of claim 4, wherein said vitamin C compound comprises ascorbyl glucoside.

6. The personal care composition of claim 3, wherein said first active comprises an avenathramide compound.

7. The personal care composition of claim 5, wherein said first active comprises an avenathramide compound.

8. The personal care composition of claim 6, wherein said avenathramide compound is selected from the group consisting of dihydrovenanthramide D, dihydrovenanthramide E, and combinations thereof.

9. The personal care composition of claim 7, wherein said avenathramide compound is selected from the group consisting of dihydrovenanthramide D, dihydrovenanthramide E, and combinations thereof.

10. The personal care composition of claim 3, wherein said first active comprises octadecenedioic acid.

11. The personal care composition of claim 5, wherein said first active comprises octadecenedioic acid.

12. The personal care composition of claim 10, wherein said octadecenedioic acid comprises octadecenedioic acid having a double bond between carbons 8 and 9.

13. The personal care composition of claim 11, wherein said octadecenedioic acid comprises octadecenedioic acid having a double bond between carbons 8 and 9.

14. The personal care composition of claim 3, wherein said first active comprises methyl sulfonyl methane.

15. The personal care composition of claim 5, wherein said first active comprises methyl sulfonyl methane.
